(19) United States
(12) Patent Application Publication
(10) Pub. No.: US 2024/0140955 A1
(43) Pub. Date: May 2, 2024

(54) PEG-MODIFIED CYCLIC DIPEPTIDES

(71) Applicant: NOVMETAPHARMA CO., LTD., Seoul (KR)

(72) Inventors: Heon Jong LEE, Incheon-si (KR); Hoe Yune JUNG, Pohang-si (KR)

(73) Assignee: NOVMETAPHARMA CO., LTD., Seoul (KR)

(21) Appl. No.: 18/476,630

(22) Filed: Sep. 28, 2023

Related U.S. Application Data
(60) Provisional application No. 63/411,508, filed on Sep. 29, 2022.

(51) Int. Cl. C07D 487/06

(52) U.S. Cl. CPC C07D 487/06

(57) ABSTRACT

Novel PEG modified cyclic dipeptide compounds are disclosed. The modified cyclic dipeptide compounds contain a cyclic dipeptide and a biocompatible polymer attached to the cyclic dipeptide. The cyclic dipeptide may be proline-containing, hydroxyproline-, or histidine-containing cyclic dipeptide. In one embodiment, the cyclic dipeptide is a cyclo (-his-pro). Exemplary modified cyclic dipeptide compounds are represented by Formula A′:

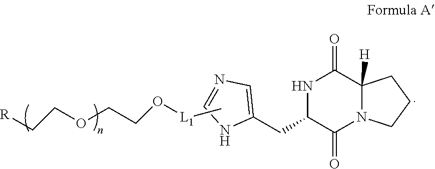

### CROSS REFERENCE TO RELATED APPLICATIONS

This application claims benefits of and priority from U.S. Provisional Application No. 63/411,508 filed Sep. 29, 2022, of which the content is incorporated by reference in its entirety.

### TECHNICAL FIELD

The present disclosure is directed to novel modified cyclic dipeptide compounds.

### BACKGROUND

A cyclic dipeptide is a cyclic peptide containing 2,5-diketopiperazine rings. Cyclic dipeptides are reported have advantages of structures and properties, including excellent hydrogen-bonding-forming ability, structural rigidity, and enzymatic stability. In cyclic dipeptides, since two amino acids are bound at the terminal portions via an amide, cyclic dipeptides have a high solubility to lipid, compared to a linear dipeptide (particularly, a linear dipeptide having the same amino acid constitution) having exposed polar groups such as a terminal carboxyl group and amino group. Thus, cyclic dipeptides may be excellent in permeability through the digestive tract and the membrane, compared to corresponding linear dipeptides.

For example, Cyclo (-His-Pro), C₁₁H₁₄N₄O₂, has been known as an anhydrous dipeptide having the CAS Registry Number 53109-32-3. It is an endogenous cyclic dipeptide derived in vivo from the hydrolytic removal of the amino-terminal pyroglutamic acid residue of the hypothalamic thyrotropin-releasing hormone. Cyclo (-His-Pro) can all be synthesized ex-vivo by conventional chemical methods. It may be important in regulating the nature of the glial cell contribution. Grotelli et al., The Role of Cyclo(His-Pro) in Neurodegeneration, Int J Mol Sci. 2016 August; 17(8): 1332. Cyclo (His-Pro) is ubiquitous in the central nervous system and is a key substrate of organic cation transporters, which are strongly linked to neuroprotection. The cyclic dipeptide can also cross the brain-blood-barrier and, once in the brain, can affect diverse inflammatory and stress responses by modifying the Nrf2-NF-κB signaling axis.

Cyclo(Leu-Gly) is reported to have a memory function-improving action and cyclo(Asp-Pro) is reported to have a suppressing action on preference for fat.

Cyclo(Trp-Pro) has been reported to have an anticancer action, cyclo(His-Pro) and cyclo(Gly-Pro) have been reported to have an antibacterial action, cyclo(Gly-Pro) to have a memory function-improving action, and cyclo(Tyr-Pro) and cyclo(Phe-Pro) to have a biological herbicide action. Alan D. Borthwick, Chem. Rev. 2012, 112, 7, 3641-3716.

The present inventors performed researches to improve chemical and physical properties of cyclic dipeptide compounds. Therefore, the present disclosure is directed to new cyclic dipeptide conjugates with improved characteristics.

### SUMMARY

In an aspect, a cyclic dipeptide conjugate that can be obtained by coupling a biocompatible polymer to a cyclic dipeptide is disclosed. Throughout the present disclosure, the term “modified cyclic dipeptide(s)” and “PEG modified cyclic dipeptide(s)” are interchangeably used, unless specified otherwise.

In some aspects, the modified cyclic dipeptide compounds may be represented by the following formula (A):

(X₁—X₂)-(L₁)ₚ-O— [symbol] CH₂CH₂O)ₙ-(L₂)_{q}-R Formula (A)

wherein
X₁—X₂ is a cyclic dipeptide;
L₁ is a linker selected from C1-C6 alkyl, C1-C6 alkyl-C(O), —C(O)—, —S(O)—, —S(O)₂—, —P(O)(O⁻)— or C(O)—C1-C6 alkyl;
L₂ is C1-C6 alkyl or C1-C6 alkyl-C(O);
R is hydrogen, a linear or branched C1-C6 alkyl, a linear or branched C1-C6 alkoxy, or a C6-10 aryl;
p is an integer 0 or 1;
q is an integer 0 or 1; and
n is 1 to 500.

In an aspect, the cyclic dipeptide X1-X2 may be a proline (Pro)-containing cyclic dipeptide, hydroxyproline (Hyp)-containing cyclic dipeptide, or histidine-containing cyclic dipeptide. In some embodiments, the proline-containing cyclic dipeptide, hydroxyproline-containing cyclic dipeptide, or histidine-containing cyclic dipeptide may be represented by X1-Pro, X1-Hyp, Pro-X2, Hyp-X2, X1-His, His-X2-, wherein X1 and X2 are each independently arginine, lysine, histidine, phenylalanine, tyrosine, leucine, isoleucine, methionine, valine, alanine, glycine, proline, glutamic acid, glutamine, serine, threonine, aspartic acid, asparagine, tryptophan, cysteine. In some embodiments, X1 and X2 are each independently glycine, phenylalanine, lysine, tryptophan, aspartic acid, tyrosine, leucine, histidine, proline, alanine, hydroxyproline, or arginine. In some embodiments, the cyclic dipeptide X1-X2 can be cyclo(Gly-Pro), cyclo(Phe-Pro), cyclo(Pro-Trp), cyclo(Leu-Pro), cyclo(His-Pro), cyclo(Pro-Asp), cyclo(Pro-Lys), cyclo(Pro-Tyr), cyclo(Pro-Gly), cyclo(Pro-Hyp), cyclo(Pro-Ala), cyclo(Pro-Arg), cyclo(Pro-Leu), cyclo(Hyp-Gly), cyclo(His-Leu), cyclo(Vla-His), cyclo(Met-His), cyclo(Ile-His), cyclo(His-Tyr), cyclo(His-Phe), cyclo(Gln-His), cyclo(Ala-Leu), cyclo(Trp-His), cyclo(Gly-His), cyclo(Thr-His), cyclo(Asp-His), cyclo(Asn-His), cyclo(Arg-His), a salt thereof, or an isomer thereof, or a salt thereof, or an isomer thereof.

In exemplary embodiments, the cyclic dipeptide may be cyclo(-His-Pro) or a salt thereof.

In an aspect, the biocompatible polymer comprises, but is not limited to, polyethylene glycol, polypropylene glycol, an ethylene glycol-propylene glycol copolymer, polyoxyethylated polyol, polyvinyl alcohol, a polysaccharide, dextran, or polyvinyl ethyl ether. In exemplary embodiments, the biocompatible polymer is a polyethylene glycol (PEG). In exemplary embodiments, the PEG may have a average molecular weight ranging from about 200 Da to about 25000 Da. In some embodiments, PEG may have average molecular weight ranging from about 200 Da to about 10000 Da, about 200 Da to about 7500 Da, or about 300 Da to 5000 Da.

In exemplary embodiments, the PEG moiety may be directly bonded to a nitrogen atom of histidine of the cyclic dipeptide. In exemplary embodiments, the PEG moiety may be bonded via a linker to a nitrogen atom of histidine of the cyclic dipeptide. In these exemplary embodiments, the linker may be hydrolysable so that the modified cyclic dipeptide may serve as a prodrug and, upon ingestion, converted to an active cyclic dipeptide; or the linker may be non-hydrolysable and the modified may prolong a half-life of active cyclic dipeptide in the body. In non-limiting embodiments, the hydrolysable group may be a carboxy (—O—C(O)— or —C(O)—O—) or an alkyl carboxy.

In another aspect, a method for preparing the modified cyclic dipeptide compound of Formula (A), an isomer, or a pharmaceutically acceptable salt is disclosed.

In embodiment 1, a compound of Formula A′ is disclosed:

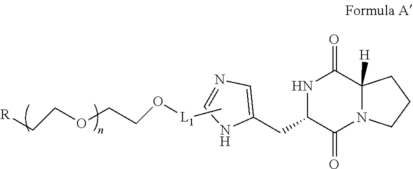

wherein
L₁ is —C(O)—, —S(O)—, —S(O)₂—, —P(O)(O⁻)—, —CH₂C(O)—, —C(O)CH₂—, —C(O)CH₂CH₂—, —CH₂—, —CH₂CH₂—, or —CH₂CH₂CH₂—, said L₁ binds to one of two nitrogen atoms of imidazole group;
n is an integer of 1-500; and
R is hydrogen, a linear or branched C1-C4 alkyl, or a linear or branched C1-C4 alkoxy, or an isomer or a pharmaceutically acceptable salt thereof.

In embodiment 2, wherein the compound of the Formula A′ is a compound of Formula A-1:

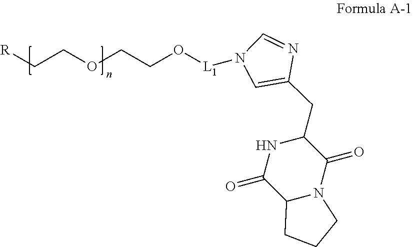

wherein, L₁, n, and R have the same meanings as defined in embodiment 1.

In embodiment 3, compounds according to embodiment 1 or 2 are disclosed, wherein n is 2-50, 3-50, 4-50, 5-50, 6-50, 7-50, 8-50, 9-50, 10-50, 1-40, 2-40, 3-40, 4-40, 5-40, 6-40, 7-40, 8-40, 9-40, 10-40, 1-30, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 2-9, 3-9, 4-9, 5- 9, 6-9, 7-9, or an isomer or a pharmaceutically acceptable salt thereof.

In embodiment 4, compounds of any one of embodiments 1-3 are disclosed, wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 4, 48, 49, or 50, or an isomer or a pharmaceutically acceptable salt thereof.

In embodiment 5, the compounds of any one of embodiments 1-4 are disclosed, wherein L₁ is —C(O)—, —S(O)—, —S(O)₂—, —P(O)(O⁻)—, —CH₂—, or —CH₂CH₂—, or an isomer or a pharmaceutically acceptable salt thereof.

In embodiment 6, compounds of any one of embodiments 1-5 are disclosed, wherein R is a linear or branched C1-C4 alkyl, or a linear or branched C1-C4 alkoxy, or an isomer or a pharmaceutically acceptable salt thereof.

In embodiment 7, a composition comprising a compound of any one of embodiments 1-6 is disclosed.

In embodiment 8, a modified cyclic dipeptide compound, represented by the following formula (A):

(X₁—X₂)-(L₁)ₚO—(CH₂CH₂O)ₙ-(L₂)_{q}-R Formula (A)

wherein
X₁—X₂ is a cyclic dipeptide;
L₁is a linker selected from C1-C6 alkyl, C1-C6 alkyl-C(O), —C(O)—, —S(O)—, —S(O)₂—, —P(O)(O⁻)—, or C(O)—C1-C6 alkyl;
L₂ is C1-C6 alkyl or C1-C6 alkyl-C(O);
R is hydrogen, a linear or branched C1-C6 alkyl, a linear or branched C1-C6 alkoxy, or a C6-10 aryl;
p is an integer 0 or 1;
q is an integer 0 or 1; and
n is 1 to 500, is disclosed.

In embodiment 9, the modified cyclic dipeptide compound according to embodiment 8 is disclosed, wherein the cyclic dipeptide may be a proline-containing cyclic dipeptide, a hydroxyroline-containing cyclic dipeptide, or histidine-containing cyclic dipeptide.

In embodiment 10, the modified cyclic dipeptide compound according to embodiment 8 or 9 are disclosed, wherein the proline-containing cyclic dipeptide, hydroxyproline-containing cyclic dipeptide, or histidine-containing cyclic dipeptide may be represented by X₁-Pro, X₁-Hyp, Pro-X₂, Hyp-X₂, His-X₂—, wherein X₁ and X₂ are each independently arginine, lysine, histidine, phenylalanine, tyrosine, leucine, isoleucine, methionine, valine, alanine, glycine, proline, glutamic acid, glutamine, serine, threonine, aspartic acid, asparagine, tryptophan, cysteine. In some embodiments, X₁ and X₂ are each independently glycine, phenylalanine, lysine, tryptophan, aspartic acid, tyrosine, leucine, histidine, proline, alanine, hydroxyproline, or arginine. In some embodiments, the cyclic dipeptide X₁-X₂ can be cyclo(Gly-Pro), cyclo(Phe-Pro), cyclo(Pro-Trp), cyclo(Leu-Pro), cyclo(His-Pro), cyclo(Pro-Asp), cyclo(Pro-Lys), cyclo(Pro-Tyr), cyclo(Pro-Gly), cyclo(Pro-Hyp), cyclo(Pro-Ala), cyclo(Pro-Arg), cyclo(Pro-Leu), cyclo(Hyp-Gly), cyclo(His-Leu), cyclo(Vla-His), cyclo(Met-His), cyclo(Ile-His), cyclo(His-Tyr), cyclo(His-Phe), cyclo(Gln-His), cyclo(Ala-Leu), cyclo(Trp-His), cyclo(Gly-His), cyclo(Thr-His), cyclo(Asp-His), cyclo(Asn-His), cyclo(Arg-His), a salt thereof, or an isomer thereof. or a salt thereof, or an isomer thereof.

### BRIEF DESCRIPTION OF THE DRAWINGS

FIG. 1 illustrates exemplary modified CHP compound (Compound 1) obtained in Example 2, in which the PEG is attached to a nitrogen atom of imidazole ring of the CHP.

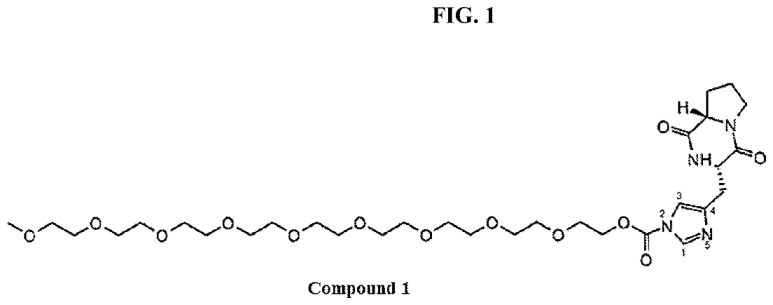

FIG. 2 illustrates exemplary modified CHP compound (Compound 2) obtained in Example 3, in which the PEG is attached to a nitrogen atom of imidazole ring of the CHP.

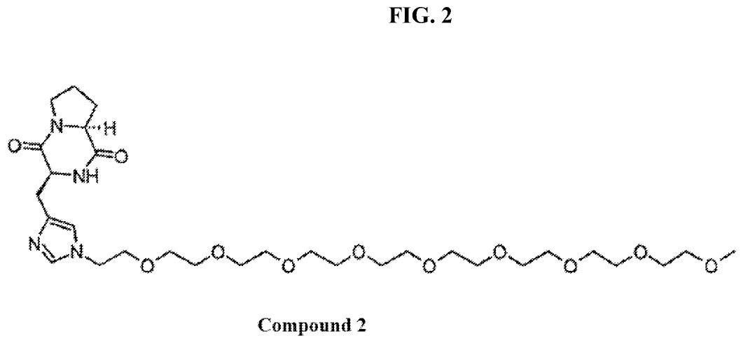

FIG. 3 shows LC-MS spectrogram of the modified CHP compound 1 obtained in Example 2.

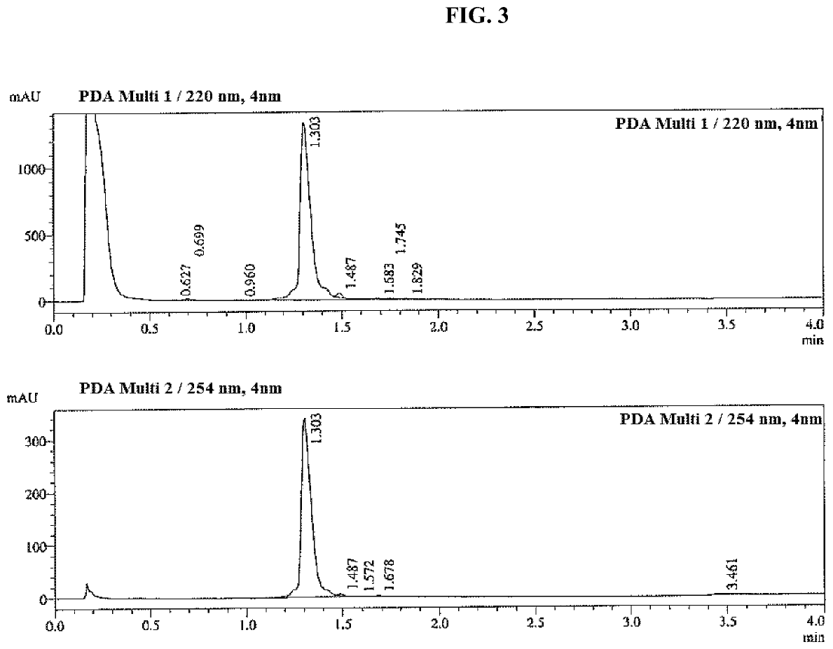

FIG. 4 shows LC-MS spectrogram of the modified CHP compound 2 obtained in Example 3.

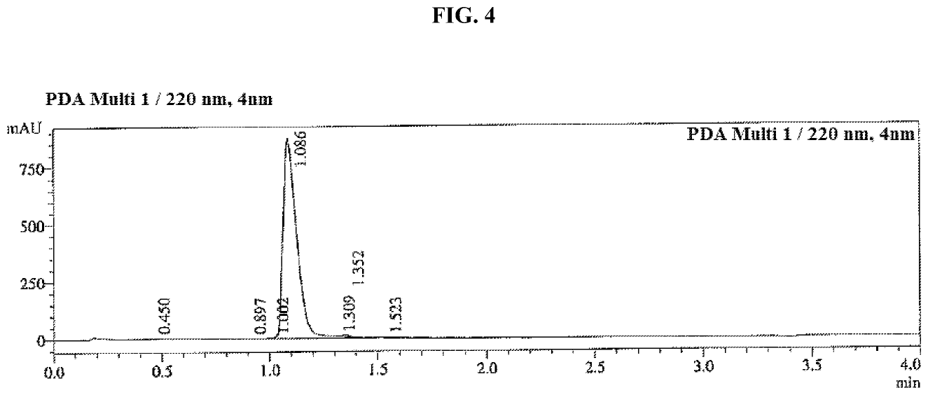

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

### Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein. In an aspect, the word “about” as used in referring to a numerical value is intended to include 10%, 9%, 8%, 7%, 6%, 5%, 4%, 3%, 2%, or 1% variance from the numerical value(s).

The term “active ingredient” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The phrase “consists essentially of” used herein with regard to a composition or formulation means that the composition or formulation contains the listed compound(s) as sole active ingredient(s) and may additionally contain a pharmaceutically acceptable inert additive(s), excipient(s), or carrier(s). Such inert additives, excipients, or carriers are known in the art.

The phrase “pharmaceutically acceptable” additives, excipients, or carriers as used herein include those well known in the art. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In the present specification, unless otherwise specified, examples of the “C1-C6 alkyl group” include methylethyl, propyl, isopropyl, butyl, isobutyl, sec-butyl, tert-butyl, pentyl, isopentyl, neopentyl, tert-pentyl3-methylbutyl, 1,2-dimethyl propyl, 1-ethylpropyl, hexyl, isohexyl, 1-methylpentyl, 2-methylpentyl, 3-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, cyclopropylmethyl, cyclobutylmethyl, cyclopentylmethyl, 1-cyclopropylethyl, 2-cyclopropylethyl, 2-cyclobutylethyl, and 2-methylcyclopropyl. Examples of the “C1-C4 alkyl group” include methylethyl, propyl, isopropyl, butyl, isobutyl, sec-butyl, tert-butyl, cyclopropyl, cyclobutyl, and cyclopropylmethyl.

As used herein, the term “conjugate” or “conjugated compound” refers to at least two components, a first component and a second component, that are generally covalently bound.

In the present specification, unless otherwise specified, the term “alkoxyl group” means a group in which the above described “alkyl group” is substituted with an oxygen atom. The alkoxyl group is generally represented by RO— (R=alkyl group).

### Modified Cyclic Dipeptide Compounds

The modified cyclic dipeptide compounds comprise a cyclic dipeptide and a biocompatible polymer liked to the cyclic dipeptide, directly or via a linker (L1).

In non-limiting embodiments, a biocompatible polymer may be polyethylene glycol (PEG). In these embodiments, the modified cyclic dipeptide compounds may be represented by the following formula (A):

(X₁—X₂)-(L₁)ₚ-O— [symbol] CH₂CH₂O)ₙ-(L₂)_{q}-R Formula (A)

wherein
X₁—X₂ is a cyclic dipeptide;
L₁ is a linker selected from C1-C6 alkyl, C1-C6 alkyl-C(O), C(O), —S(O)—, —S(O)₂—, —P(O)(O⁻)— or C(O)—C1-C6 alkyl;
L₂ is C1-C6 alkyl or C1-C6 alkyl-C(O);
R is hydrogen, a linear or branched C1-C6 alkyl, a linear or branched C1-C6 alkoxy, or a C6-10 aryl;
p is an integer 0 or 1;
q is an integer 0 or 1;
n is 1 to 500.

In some embodiments, L1 is selected from C1-C4 alkyl, C1-C4 alkyl-C(O), C(O), —S(O)—, —S(O)₂—, —P(O)(O⁻)—, or C(O)-C1-C4 alkyl.

In some embodiments, L₂ is C1-C4 alkyl or C1-C4 alkyl-C(O).

In some embodiments, R is a linear or branched C1-C4 alkyl or a linear or branched C1-C4 alkoxy.

The present disclosure encompasses an isomer (optical isomer or diastereomer) or a pharmaceutically acceptable salt of the compound described herein, unless indicated otherwise.

In exemplary embodiments, X₁—X₂ may be a proline-containing cyclic dipeptide, hydroxyproline-containing cyclic dipeptide, or histidine-containing cyclic dipeptide. In some embodiments, proline-containing cyclic dipeptide, hydroxyproline-containing cyclic dipeptide, or histidine-containing cyclic dipeptide may be represented by X₁-Pro, X₁-Hyp, Pro-X₂, Hyp-X₂, X₁-His, His-X₂-, wherein X₁ and X₂ are each independently arginine, lysine, histidine, phenylalanine, tyrosine, leucine, isoleucine, methionine, valine, alanine, glycine, proline, glutamic acid, glutamine, serine, threonine, aspartic acid, asparagine, tryptophan, cysteine. In some embodiments, X₁ and X₂ are each independently glycine, phenylalanine, lysine, tryptophan, aspartic acid, tyrosine, leucine, histidine, proline, alanine, hydroxyproline, or arginine. In some embodiments, the cyclic dipeptide X₁-X₂ can be cyclo(Gly-Pro), cyclo(Phe-Pro), cyclo(Pro-Trp), cyclo(Leu-Pro), cyclo(His-Pro), cyclo(Pro-Asp), cyclo(Pro-Lys), cyclo(Pro-Tyr), cyclo(Pro-Gly), cyclo(Pro-Hyp), cyclo(Pro-Ala), cyclo(Pro-Arg), cyclo(Pro-Leu), cyclo(Hyp-Gly), cyclo(His-Leu), cyclo(Vla-His), cyclo(Met-His), cyclo(Ile-His), cyclo(His-Tyr), cyclo(His-Phe), cyclo(Gln-His), cyclo(Ala-Leu), cyclo(Trp-His), cyclo(Gly-His), cyclo(Thr-His), cyclo(Asp-His), cyclo(Asn-His), cyclo(Arg-His), a salt thereof, or an isomer thereof.

According to some embodiments, a compound of Formula A′:

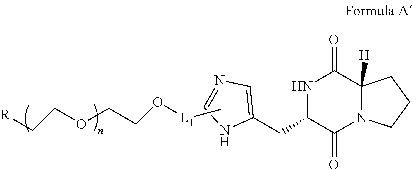

wherein
L₁ is —C(O)—, —S(O)—, —S(O)₂—, —P(O)(O⁻)—, —CH₂C(O)—, —C(O)CH₂—, —C(O)CH₂CH₂—, —CH₂—, —CH₂CH₂—, or —CH₂CH₂CH₂—, said L₁ binds to one of two nitrogen atoms of imidazole group;
n is an integer of 1-500; and
R is hydrogen, a linear or branched C1-C4 alkyl, or a linear or branched C1-C4 alkoxy, or an isomer or a pharmaceutically acceptable salt thereof are disclosed.

The present disclosure encompasses an isomer (optical isomer or diastereomer) or a pharmaceutically acceptable salt of the compound described herein, unless indicated otherwise.

In some embodiments, the modified cyclic dipeptide compounds include compounds of Formula A-1:

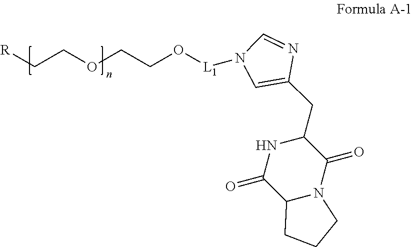

wherein L₁ may be —C(O)—, —S(O)—, —S(O)₂—, —P(O)(O⁻)—, —CH₂C(O)—, —C(O)CH₂—, —C(O)CH₂CH₂—, —CH₂—, —CH₂CH₂—, or —CH₂CH₂CH₂—, n is an integer of 1-500, 5-500, 5-400, 5-300, 5-200, 5-100, 1-100, or 1-50, and R is hydrogen, a linear or branched C1-C4 alkyl, or a linear or branched C1-C4 alkoxy. In some embodiments, n ranges about 1-50, 2-50, 3-50, 4-50, 5-50, 6-50, 7-50, 8-50, 9-50, 10-50, 1-40, 2-40, 3-40, 4-40, 5-40, 6-40, 7-40, 8-40, 9-40, 10-40, 1-30, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 8-10, or 9-10. In some particular embodiments, in the above compounds, R is a linear or branched C1-C4 alkyl, or a linear or branched C1-C4 alkoxy, and n is 1-500, 5-500, 5-400, 5-300, 5-200, 5-100, 1-50, 2-40, 3-40, 4-40, 5-40, 6-40, 7-40, 8-40, 9-40, 10-40, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, or 7-8. In some embodiments, n may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100.

In some embodiments, the modified cyclic dipeptide compounds include:

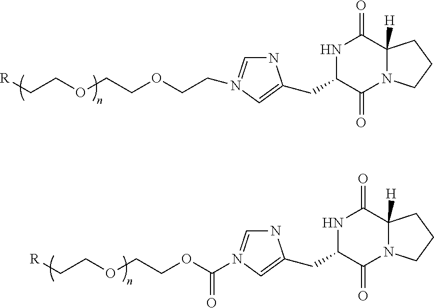

wherein R is hydrogen, a linear or branched C1-C6 alkyl, or a linear or branched C1-C6 alkoxy, and n is 1-500, or an isomer or a pharmaceutically acceptable salt thereof. In some embodiments, n ranges about 1-500, 5-500, 5-400, 5-300, 5-200, 5-100, 1-100, 1-50, 2-50, 3-50, 4-50, 5-50, 6-50, 7-50, 8-50, 9-50, 10-50, 1-40, 2-40, 3-40, 4-40, 5-40, 6-40, 7-40, 8-40, 9-40, 10-40, 1-30, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 8-10, or 9-10. In some particular embodiments, in the above compounds, R is a linear or branched C1-C4 alkyl, or a linear or branched C1-C4 alkoxy, and n is 1-500, 5-500, 5-400, 5-300, 5-200, 5-100, 1-100, 1-50, 2-40, 3-40, 4-40, 5-40, 6-40, 7-40, 8-40, 9-40, 10-40, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, or 7-8. In some embodiments, n may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100.

In some other embodiments, the modified cyclic dipeptide compounds include:

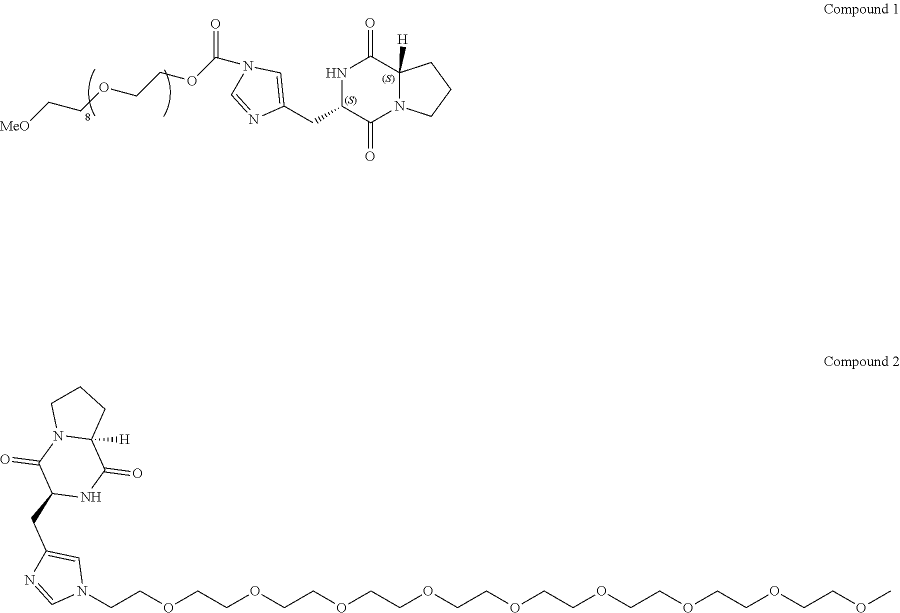

wherein n is 1-500, or an isomer or a pharmaceutically acceptable salt thereof. In some embodiments of the compounds, n ranges about 1-500, 5-500, 5-400, 5-300, 5-200, 5-100, 1-100, 1-50, 2-50, 3-50, 4-50, 5-50, 6-50, 7-50, 8-50, 9-50, 10-50, 1-40, 2-40, 3-40, 4-40, 5-40, 6-40, 7-40, 8-40, 9-40, 10-40, 1-30, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, or 7-8. In some embodiments, n may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100.

In some embodiments, the modified cyclic dipeptide compounds may be a dimer comprising two cyclic dipeptides, which may be same or different, linked to both ends of the polyethylene glycol. An exemplary embodiment of such a dimer includes, but is not limited to:

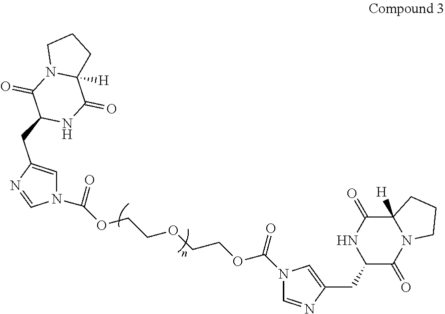

wherein all the symbols are same as defined in above.

While not wishing to be bound by theory, it is believed that the modified cyclic dipeptide according to the present disclosure may be expected to have improved in vivo stability, stronger biological activity, longer half-life and reduced frequency of medication, less enzymatic degradation, better solubility, enhanced permeability and improved metabolic clearance profile. For example, the modified cyclic dipeptides (e.g., compound 1 or compound 5) having a hydrolysable ester (for example, —C(O)O—) between the polymer and the cyclic dipeptide may function as a prodrug and, upon administration to a subject, can release the active cyclic dipeptide by enzymatic cleavage in the body. On the other hand, the modified cyclic dipeptides (e.g., compound 3) without a hydrolysable bond in the molecule may not undergo cleavage in the body and functions as a new chemical entity which may be able to exhibit additional or new physiological activities.

The modified cyclic dipeptide compounds may be prepared by linking a biocompatible polymer to the cyclic dipeptide, either directly or via a linker (L₁). The biocompatible polymer may have a reactive group (or an activating group) for covalent bonding to the cyclic dipeptide, as described below. In certain embodiments, the biocompatible polymer suitable for modifying a cyclic dipeptide may be a functional form having a reactive group at one or both ends. Reactive groups may be a carboxylic acid (e.g., polyethylene glycol diacid (i.e., PEG having carboxylic acid at both ends) which can be used for preparing a dimer), a tosyl group, an aldehyde group, a propione aldehyde group, a butyl aldehyde group, a maleimide group, succinimidyl propionate, succinimidyl carboxymethyl, hydroxy succinimidyl, or succinimidyl carbonate. As described below, in one embodiment, a cyclic dipeptide and mPEG-OH may be reacted in the presence of a phosgene in the presence of a base known in the art to obtain a desired PEG modified cyclic dipeptide (see, Example 2). In another embodiment, when tosyl-PEG is employed to pegylate a cyclic dipeptide, the tosyl-PEG and the cyclic dipeptide may be reacted in the presence of a base such as a carbonate, preferably cesium carbonate, to obtain a desired PEG modified cyclic dipeptide (see, Example 3).

### Cyclic Dipeptides

In exemplary embodiments, a cyclic dipeptide may be a proline-containing, hydroxyproline-containing cyclic dipeptide, or histidine-containing cyclic dipeptide. In some embodiments, proline-containing cyclic dipeptide, hydroxyproline-containing cyclic dipeptide, or histidine-containing cyclic dipeptide may be represented by X₁-Pro, X₁-Hyp, Pro-X₂, Hyp-X₂, X₁-His, His-X₂—, wherein X₁ and X₂ are each independently arginine, lysine, histidine, phenylalanine, tyrosine, leucine, isoleucine, methionine, valine, alanine, glycine, proline, glutamic acid, glutamine, serine, threonine, aspartic acid, asparagine, tryptophan, cysteine. In some embodiments, X₁ and X₂ are each independently glycine, phenylalanine, lysine, tryptophan, aspartic acid, tyrosine, leucine, histidine, proline, alanine, hydroxyproline, or arginine. In some embodiments, the cyclic dipeptide X₁—X₂ can be cyclo(Gly-Pro), cyclo(Phe-Pro), cyclo(Pro-Trp), cyclo(Leu-Pro), cyclo(His-Pro), cyclo(Pro-Asp), cyclo(Pro-Lys), cyclo(Pro-Tyr), cyclo(Pro-Gly), cyclo(Pro-Hyp), cyclo(Pro-Ala), cyclo(Pro-Arg), cyclo(Pro-Leu), cyclo(Hyp-Gly), cyclo(His-Leu), cyclo(Vla-His), cyclo(Met-His), cyclo(Ile-His), cyclo(His-Tyr), cyclo(His-Phe), cyclo(Gln-His), cyclo(Ala-Leu), cyclo(Trp-His), cyclo(Gly-His), cyclo(Thr-His), cyclo(Asp-His), cyclo(Asn-His), cyclo(Arg-His), a salt thereof, or an isomer thereof. In some embodiments, cyclic dipeptide may be cyclo (his-pro) (CHP) or a salt thereof. Cyclic dipeptides may be prepared by known method which can be exemplified by, but is not limited to, the methods described in JP6826359B2.

### Biocompatible Polymer

A biocompatible polymer used to modify a cyclic dipeptide according to the instant disclosure may be a polyalkylene glycol (e.g., polyethylene glycol, polypropylene glycol, an ethylene glycol-propylene glycol copolymer, polyoxyethylated polyol), polyvinyl alcohol, a polysaccharide, dextran, or polyvinyl ethyl ether. In exemplary embodiments, the biocompatible polymer is polyalkylene glycol derivatives. In particular exemplary embodiments, the polyalkylene glycol may be polyethylene glycol (PEG) derivatives.

A variety of PEG may be used for modify (e.g., PEGylate) the cyclic dipeptides. A suitable reactive PEG reagent can be used. In some embodiments, the reactive PEG reagent will result in formation of a carbamate or amide bond upon coupling to the cyclic dipeptide at the nitrogen atom or carbon atom of the cyclic dipeptide.

In exemplary embodiments, when the polymer is PEG, for example, it may be preferred to use a methoxy-PEG (commonly referred to as mPEG), which is a form of PEG, typically linear, wherein one terminus of the polymer is a methoxy (—OCH₃) group, while the other terminus is a hydroxyl or other functional group that can be optionally chemically modified. The structure of an mPEG is given below.
CH₃O—(CH₂CH₂O)n—CH₂CH₂—, where the value of (n) is as described herein.

monofunctionally activated hydroxyPEGs (e.g., hydroxyPEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyPEG-monoiodoacetamides, hydroxyPEG-monomaleimides, hydroxyPEG-monoorthopyridyl disulfides, hydroxyPEG-monooximes, hydroxyPEG-monophenyl carbonates, hydroxyPEG-monophenyl glyoxals, hydroxyPEG-monothiazolidine-2-thiones, hydroxyPEG-monothioesters, hydroxyPEG-tosylestoers, hydroxyPEG-monothiols, hydroxyPEG-monotriazines and hydroxyPEG-monovinylsulfones) can be used to modify the cyclic dipeptide according to the present disclosure to prepare the modified cyclic dipeptide of Formula (I).

In other exemplary embodiments, mPEG-oTS, mPEG₂-NHS, mPEG₂-ALD, multi-Arm PEG, mPEG(MAL)₂, mPEG₂(MAL), mPEG-NH₂, MPEG-SPA, mPEG-SBA, mPEG-thioesters, mPEG-Double Esters, mPEG-BTC, mPEG-ButyrALD, MPEG-ACET, heterofunctional PEGs (e.g., NH₂-PEG-COOH, Boc-PEG-NHS, Fmoc-PEG-NHS, NHS-PEG-VS, NHS-PEG-MAL), PEG acrylates (ACRL-PEG-NHS), PEG-phospholipids (e.g., mPEG-DSPE), carboxyl-PEGs, carboxyl-PEG-carboxyl, p-NP-PEGs, tosyl-PEGs, Tresyl-PEGs, aldehyde PEGs, acetal-PEGs, amino-PEGs, thiol-PEGs, maleimido-PEGs, amino-PEG-COOH, succinimidyl-PEGs, carboxylic anhydride type-PEG, functionalized PEG-phospholipid, and the like may be employed.

According to embodiments discussed herein, linear and branched PEGs of various sizes are suitable, including those with average molecular weights (excluding the mass of the activating group or reactive group) in the range of about 44 Da to about 50000 Da, about 200 Da to about 50000 Da, or about 200 Da to about 22000 Da. Suitable ranges of average molecular weights include but are not limited to about 100 Da to about 25000 Da, about 100 Da to about 10000 Da; about 100 Da to about 8000 Da; about 100 Da to about 7000 Da; about 150 Da to about 6000 Da; about 200 Da to about 5000 Da, about 250 Da or about 5000 Da, about 250 Da to about 4500 Da, and about 250 Da, about 300 Da, about 350 Da, about 400 Da, about 450 Da, about 500 Da, about 750 Da, about 1000 Da, about 1500 Da, about 2000 Da, about 3000 Da, about 4000 Da, about 5000 Da, about 6000 Da, about 7000 Da, about 8000 Da, about 9000 Da, or about 10000 Da. In the case of linear PEGs, the molecular weight range of about 50 Da to about 25000 Da corresponds to a degree of polymerization (n) of the Formula (I) in the range of about 1 to about 500 monomeric units of ethylene oxide. Commercially available PEG derivatives may be used. For example, PEG 200 (n is about 4.2) having average molecular weights of 190-210 Da, PEG1000 (n is about 22.3) having average molecular weights of 900-1000 Da, PEG2000 (n=40-50) having average molecular weights of 1800-2200 Da, or PEG4000 (n=69-84) having average molecular weights of 3000-4800 or PEG4000 (n=26-32) having average molecular weights of 3600-4400 Da, and the like may be used.

In some embodiments, the degree of polymerization (n) of the Formula (I) may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300, 400, or 500, or ranges about 1-500, 5-500, 5-400, 5-300, 5-200, 5-100, 1-50, 2-50, 3-50, 4-50, 5-50, 6-50, 7-50, 8-50, 9-50, 10-50, 1-40, 2-40, 3-40, 4-40, 5-40, 6-40, 7-40, 8-40, 9-40, 10-40, 1-30, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, or 7-10.

### Compositions and Uses

The modified cyclic dipeptide compounds of Formula (I) may be comprised in a composition as an active ingredient for regulating or lowering glucose level in blood of a subject, or ameliorating or treating diabetes, anti-obesity, anti-inflammatory, and/or antioxidant effects. And, the modified cyclic dipeptide compounds of Formula (I) of present disclosure may be used in therapeutically effective amounts to treat a variety of diseases and disorders, such as metabolic diseases, neurodegenerative diseases, Alzheimer's disease, Parkinson's disease, Huntington's disease, acute kidney injury (AKI), chronic kidney disease (CKD), pulmonary fibrosis, kidney fibrosis, to provide cytoprotection against oxidative damage, to suppress inflammatory responses in the PC12 cell line, and as an appetite suppressant.

The composition according to the present disclosure may be a pharmaceutical composition.

The pharmaceutical composition according to the present disclosure may further include suitable carriers, excipients or diluents conventionally used in the manufacture of pharmaceutical compositions. In this connection, a content of the modified cyclic dipeptide or a salt or fraction thereof included in the composition may be preferably 0.0001 to 10% by weight, preferably 0.001 to 1% by weight. However, the present disclosure is not particularly limited thereto.

As used herein, the term “pharmaceutically acceptable salt” refers to a salt that can be pharmaceutically used, among the substances having cations and anions coupled by electrostatic attraction. Typically, it may include metal salts, salts with organic bases, salts with inorganic acids, salts with organic acids, salts with basic or acidic amino acids or the like. Examples of the metal salts may include alkali metal salts (sodium salts, potassium salts, etc.), alkaline earth metal salts (calcium salts, magnesium salts, barium salts, etc.), aluminum salts or the like; examples of the salts with organic bases may include salts with triethylamine, pyridine, picoline, 2,6-lutidine, ethanolamine, diethanolamine, triethanolamine, cyclohexylamine, dicyclohexylamine, N,N′-dibenzylethylenediamine or the like; examples of the salts with inorganic acids may include salts with hydrochloric acid, hydrobromic acid, nitric acid, sulfuric acid, phosphoric acid, or the like; examples of the salts with organic acids may include salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid or the like; examples of the salts with basic amino acids may include salts with arginine, lysine, ornithine or the like; and examples of the salt with acidic amino acids include salts with aspartic acid, glutamic acid or the like.

The pharmaceutical composition may be administered orally or parenterally. Extracorporeal or intraperitoneal injection, intrarectal injection, subcutaneous injection, intravenous injection, intramuscular injection or intrathoracic injection may be preferably selected for parenteral administration.

The pharmaceutical composition according to the present disclosure may further include commonly used excipients, disintegrants, sweeteners, lubricants, flavors, and the like. The disintegrant may be selected from the group consisting of sodium starch glycolate, crospovidone, croscarmellose sodium, alginic acid, carboxymethylcellulose calcium, carboxymethylcellulose sodium, chitosan, guar gum, low-substituted hydroxypropylcellulose, magnesium aluminum silicate, and polacrilin potassium. Further, the pharmaceutical composition according to the present disclosure may further include a pharmaceutically acceptable additive. In this connection, the pharmaceutically acceptable additives may include starch, gelatinized starch, microcrystalline cellulose, lactose, povidone, colloidal silicon dioxide, calcium hydrogen phosphate, lactose, mannitol, sugar, arabic gum, pregelatinized starch, corn starch, powdered cellulose, hydroxypropylcellulose, opadry, sodium starch glycolate, carnauba lead, synthetic aluminum silicate, stearic acid, magnesium stearate, aluminum stearate, calcium stearate, white sugar, dextrose, sorbitol, talc, etc. The content of one or more pharmaceutically acceptable additive(s) according to the present disclosure may be in a range of 0.1 to 90 parts by weight based on the total weight of the pharmaceutical composition.

In an embodiment, the composition comprises, or consists essentially of the modified cyclic dipeptide of Formula (I), an isomer, or a pharmaceutically acceptable salt thereof, as an active ingredient. In an embodiment, the composition comprises, or consists essentially of the modified cyclic dipeptide of Formula (I), an isomer, or a pharmaceutically acceptable salt thereof; and a zinc metal, an organic or inorganic salt of zinc, a zinc compound, or a zinc ion, as active ingredients. In an embodiment, the composition does not include zinc metal, an organic or inorganic salt of zinc, a zinc compound, or a zinc ion. In other embodiment, the composition may contain zinc metal, an organic or inorganic salt of zinc, a zinc compound, or a zinc ion.

Solid form preparations for oral administration include powders, granules, tablets, capsules, soft capsules, pills and the like. Liquid preparations for oral administration include suspensions, solutions, emulsions, syrups, aerosols and the like. The liquid preparation may include various excipients such as a wetting agent, a sweetening agent, a fragrance, and a preservative in addition to water and liquid paraffin which are commonly used simple diluents. Formulations for parenteral administration may be formulated in the form of external preparations such as powders, granules, tablets, capsules, sterilized aqueous solutions, liquid preparations, non-aqueous solvents, suspensions, emulsions, syrups, suppositories, and aerosols and sterile injectable preparation, respectively, according to conventional methods. Preferably, externally-applied skin pharmaceutical preparations such as creams, gels, patches, sprays, ointments, alerts, lotions, liniments, pastes or cataplasms may be prepared, but are not limited thereto. As the non-aqueous formulation or suspension, propylene glycol, polyethylene glycol, vegetable oil such as olive oil, injectable ester such as ethyl oleate, or the like may be used. witepsol, macrogol, tween 61, cocoa butter, laurin butter, glycerogelatin or the like may be used as a base of the suppository.

The preferred dosage of the pharmaceutical composition according to the present disclosure varies depending on the degree of absorption of the active substance in the body, inactivation and excretion rate, age, sex and condition of the patient, and severity of the disease to be treated. The dosage may be suitably selected by those skilled in the art. However, for the desired effect, in the case of oral administration, generally, the pharmaceutical composition according to the present disclosure may be administrated to one adult at a dosage of 0.0001 to 100 mg/kg, preferably 0.001 to 100 mg/kg per a day. The administration may be carried out once a day or divided into several times for one day. The dosage does not in any respect limit the scope of the present disclosure. The pharmaceutical composition according to the present disclosure has little toxicity and side effects, and may be used safely even when taken for a long time.

The composition according to the present disclosure may be a foodstuff or dietary supplement. The foodstuff or dietary supplement contains the compound of Formula (I) or an isomer or a food acceptable salt thereof. As used herein, the term “food acceptable salt” refers to a salt that can be used as food, among the substances having cations and anions coupled by electrostatic attraction. Specific examples of the food acceptable salt include examples of the above-mentioned pharmaceutically acceptable salts.

The dietary supplement, which sometimes also is referred to as in some countries as functional food, health food, or health supplement food, refers to foods that have an active health maintaining or promoting effect compared to general foods. The food and/or dietary supplement may be prepared in various forms such as tablets, capsules, powders, granules, liquids, and pills in order to obtain useful effects. As used herein, the term “functional food” is the same term as “food for special health use (FoSHU)”. This food means foods with high medical effects that have been processed so that the biocontrol function can be efficiently enabled in addition to nutrition.

As a specific example of such a health functional food, a processed food may be produced having good storage properties while converting the above-mentioned composition into foods having characteristics of agricultural products, livestock products or aquatic products.

It will be readily apparent to one of ordinary skill in the relevant arts that other suitable modifications and adaptations to the methods and applications described herein may be made without departing from the scope of the present disclosure or any embodiment thereof. The present disclosure will be more clearly understood by reference to the following examples, which are included herewith for purposes of illustration only and are not intended to be limiting of the present disclosure.

### EXAMPLES

### Abbreviations

ACN: acetonitrile
mPEG: methoxy polyethylene glycol
MC: methylene chloride
DCC: N,N′-Dicyclohexylcarbodiimide
DIEA or DIPEA: N,N-Diisopropylethylamine
DMAP: 4-dimethylaminopyridine
DMF: dimethylformamide
TEA: triethanolamine
THE: tetrahydrofuran
Ts: tosyl

### Reference Example 1: Preparation of methyl (2S)-1-[(2S)-2-(tert-butoxycarbonylamino)-3-(1H-imidazol-5-yl)propanoyl]pyrrolidine-2-carboxylate

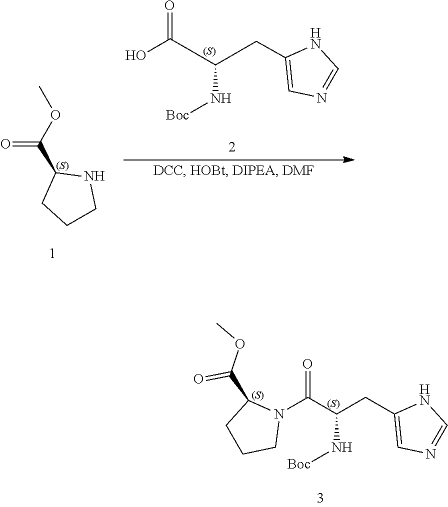

To a solution of (2S)-2-(tert-butoxycarbonylamino)-3-(1H-imidazol-5-yl)propanoic acid (50.0 g, 196 mmol) and methyl (2S)-pyrrolidine-2-carboxylate (38.9 g, 235 mmol, HCl) in DMF (500 mL) was added DCC (44.5 g, 216 mmol, 43.6 mL) and HOBt (29.1 g, 216 mmol), DIEA (101 g, 784 mmol, 137 mL). The mixture was stirred at 25° C. for 12 hrs. TLC (CH₂Cl₂/MeOH=10/1, R_{f}=0.2) showed the reaction was complete. The reaction mixture was diluted with CH₂Cl₂ (500 mL) washed with NH₄Cl 500 mL (250 mL×2). And washed with NaHCO₃ (250 mL×2). And washed with brine 500 mL (250 mL×2). The combined organic layers were dried over Na₂SO₄, filtered and concentrated under reduced pressure to give a residue, which was purified by column chromatography (SiO₂, CH₂Cl₂:MeOH=1/0 to 10/1) to give methyl (2S)-1-[(2S)-2-(tert-butoxycarbonylamino)-3-(1H-imidazol-5-yl)propanoyl]pyrrolidine-2-carboxylate (43.0 g, 55.4% yield) as a white solid.

¹HNMR: 400 MHz, DMSO-d₆ δ 7.53 (s, 1H), 7.03 (d, J=8.0 Hz, 1H), 6.79 (s, 1H), 4.37-4.43 (m, 1H), 4.30-4.35 (m, 1H), 4.12 (s, 1H), 3.58-3.61 (m, 3H), 3.17 (s, 2H), 2.68-2.78 (m, 2H), 2.11-2.21 (m, 1H), 1.80-1.84 (m, 2H), 1.33 (s, 9H).

LCMS: m/z=367.1 (M+H)⁺, Rt=0.406 min.

### Reference Example 2: Preparation of methyl (2S)-1-[(2S)-2-amino-3-(1H-imidazol-5-yl)propanoyl]pyrrolidine-2-carboxylate

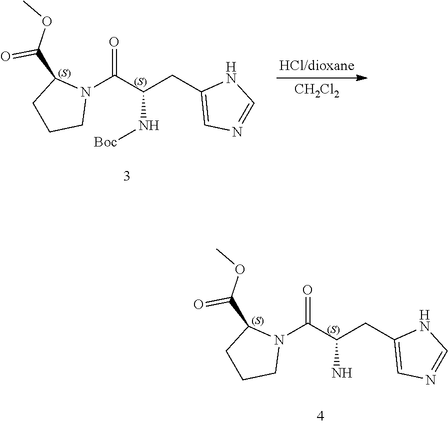

To a solution of methyl (2S)-1-[(2S)-2-(tert-butoxycarbonylamino)-3-(1H-imidazol-5-yl)propanoyl]pyrrolidine-2-carboxylate (43.0 g, 117 mmol) in CH₂Cl₂ (400 mL) was added HCl/dioxane (4 M, 100 mL). The mixture was stirred at 25° C. for 2 hrs. TLC (CH₂Cl₂/MeOH=20/1, R_{f}=0.3) showed the reaction was complete. The reaction mixture was concentrated under reduced pressure to give a methyl (2S)-1-[(2S)-2-amino-3-(1H-imidazol-5-yl)propanoyl]pyrrolidine-2-carboxylate (35.5 g, 91.7% yield, HCl) as a white solid.

¹HNMR: 400 MHz, CD₃OD δ 8.97 (d, J=1.2 Hz, 1H), 7.60 (d, J=1.2 Hz, 1H), 4.69 (t, J=6.8 Hz, 1H), 4.57 (dd, J=9.2, 5.6 Hz, 1H), 3.79-3.86 (m, 2H), 3.76 (s, 3H), 3.66 (s, 5H), 3.47-3.52 (m, 1H), 3.42 (dd, J=6.8, 1.6 Hz, 1H), 2.30-2.36 (m, 1H), 1.99-2.08 (m, 3H).

### Reference Example 3: Preparation of (3S,8aS)-3-(1H-imidazol-5-ylmethyl)-2,3,6,7,8,8a-hexahydropyrrolo[1,2-a]pyrazine-1,4-dione

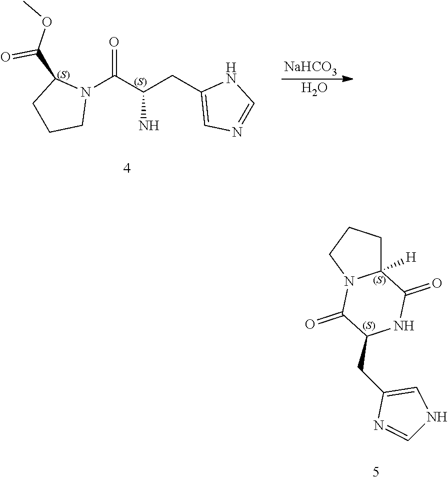

To a solution of methyl (2S)-1-[(2S)-2-amino-3-(1H-imidazol-5-yl)propanoyl]pyrrolidine-2-carboxylate (35.5 g, 117 mmol, HCl) and NaHCO₃ (9.85 g, 117 mmol, 4.56 mL) in H₂O (3 L). The mixture was stirred at 130° C. for 2.5 hrs. TLC (CH₂Cl₂/MeOH=10/1, R_{f}=0.3) showed the reaction was complete. After filtration via filter paper Spin dry add methanol and then spin dry again the water layer was concentrated under reduced pressure to dryness to give by column chromatography (SiO₂, CH₂Cl₂/MeOH=1/0 to 10/1) (3S,8aS)-3(1H-imidazol-5-ylmethyl)-2,3,6,7,8,8a-hexahydropyrrolo[1,2-a]pyrazine-1,4-dione (25.5 g, 90.9% yield,) as a colorless gum.

LCMS: m/z=235.2 (M+H)⁺, Rt=0.273 min.

### Example 1: Preparation of 2-[2-[2-[2-[2-[2-[2-[2-(2-methoxyethoxy)ethoxy]ethoxy]ethoxy]ethoxy]ethoxy]ethoxy]ethoxy]ethyl 4-methylbenzenesulfonate

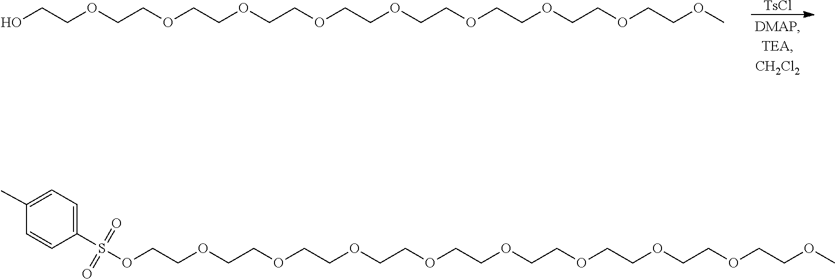

To a solution of 2-[2-[2-[2-[2-[2-[2-[2-(2-methoxyethoxy)ethoxy]ethoxy]ethoxy]ethoxy]ethoxy]ethoxy]ethoxy]ethanol (4.0 g, 9.33 mmol) in CH₂Cl₂ (40 mL) was added TEA (2.83 g, 28.0 mmol, 3.90 mL), DMAP (114 mg, 933 umol) and 4-methylbenzenesulfonyl chloride (2.67 g, 14.0 mmol). The mixture was stirred at 25° C. for 2 hrs. TLC (CH₂Cl₂/MeOH=10/1, R_{f}=0.4) showed the reaction was complete. The reaction mixture was diluted with CH₂Cl₂ (50 mL) and washed with brine (25 mL×2). The combined organic layers were dried over Na₂SO₄, filtered and concentrated under reduced pressure to give a residue as yellow solid, which was purified by column chromatography (SiO₂, CH₂Cl₂/MeOH=1/0 to 10/1) to give 2-[2-[2-[2-[2-[2-[2-[2-(2-methoxyethoxy)ethoxy]ethoxy]ethoxy]ethoxy]ethoxy]ethoxy]ethoxy]ethyl 4-methylbenzenesulfonate (4.2 g, 54.2% yield) as a yellow solid.

¹HNMR (400 MHz, CD₃OD) δ 7.76 (d, J=8.0 Hz, 2H), 7.41 (br d, J=8.0 Hz, 2H), 4.11 (t, J=4.4 Hz, 2H), 3.54-3.67 (m, 32H), 3.45-3.53 (m, 7H), 2.42 (s, 3H)

LCMS m/z=583.7 (M+H)⁺, Rt=1.218 min

### Example 2: Preparation of Compound 1

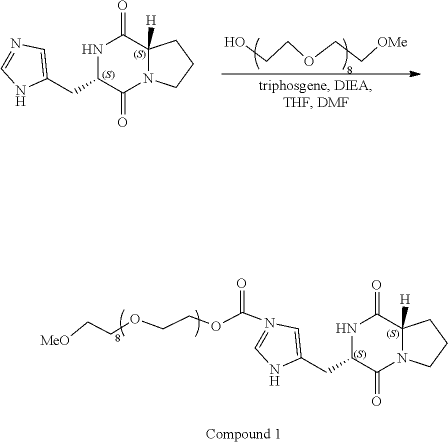

To a solution of triphosgene (1.66 g, 5.60 mmol) in TRF (15 mL) was added DIEA (1.81 g, 14.0 mmol, 2.44 mL) and 2-[2-[2-[2-[2-[2-[2-[2-(2-methoxyethoxy)ethoxy]ethoxy]ethoxy]ethoxy]ethoxy]ethoxy]ethoxy]ethanol (3.00 g, 7.00 mmol) in TRF (15 mL) dropwise at 0° C. and the mixture was stirred at 0° C. for 30 mins. And then (3S,8aS)-3-(1H-imidazol-5-ylmethyl)-2,3,6,7,8,8a-hexahydropyrrolo[1,2-a]pyrazine-1,4-dione (1.64 g, 7.00 mmol) in DMF (15 mL) was added to the mixture. The final mixture was stirred at 25° C. for 10 hrs. TLC (CH₂Cl₂/MeOH=10:1, Rf=0.3, I2) showed the reaction was complete. The reaction mixture was quenched by addition H₂O (50 mL) at 0° C. and extracted with CH₂Cl₂ (50 mL×3). The combined organic layers were washed with brine (50 mL×3), dried over Na₂SO₄, filtered and concentrated under reduced pressure to give a residue, which was purified by prep-HPLC (column: WELCH XTIMATE™ C18 150*40 mm*10 um; mobile phase: [Water-ACN]; B %: 0%-36%, 30 min). to give 2-[2-[2-[2-[2-[2-[2-[2-(2-methoxyethoxy)ethoxy]ethoxy]ethoxy]ethoxy]ethoxy]ethoxy]ethoxy]ethyl 4-[[(3S,8aS)-1,4-dioxo-2,3,6,7,8, 8a-hexahydropyrrolo[1,2-a]pyrazin-3-yl]methyl]imidazole-1-carboxylate (Compound 1) (1.35 g, 72.75% yield, 97% purity) as a yellow oil.

¹HNMR: δ (d, J=1.2 Hz, 1H), 7.91 (s, 1H), 7.39 (s, 1H), 4.47-4.49 (m, 2H), 4.30-4.35 (m, 1H), 4.17-4.22 (m, 1H), 3.73-3.76 (m, 2H), 3.56-3.59 (m, 2H), 3.48-3.51 (m, 30H), 3.41-3.43 (m, 2H), 3.23 (s, 3H), 3.15 (dd, J=15.2 4.4 Hz, 1H), 2.73 (dd, J=15.2, 7.6 Hz, 1H), 2.09-2.15 (m, 1H), 2.07 (s, 1H), 1.79-1.87 (m, 3H).

### Example 3: Preparation of Compound 2

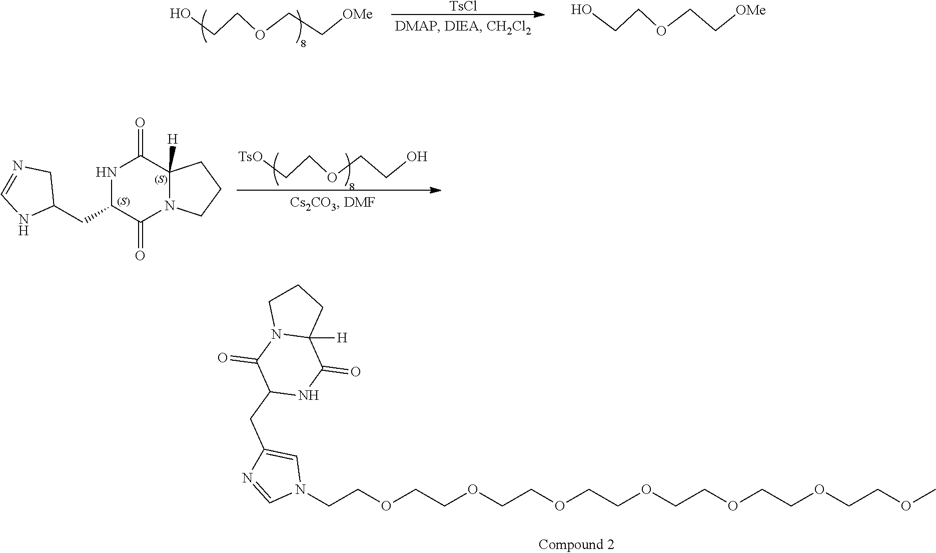

3-1. Preparation of 2-[2-[2-[2-[2-[2-[2-[2-(2-methoxyethoxy)ethoxy]ethoxy]ethoxy]ethoxy]ethoxy]ethoxy]ethoxy]ethyl 4-methylbenzenesulfonate

To a solution of 2-[2-[2-[2-[2-[2-[2-[2-(2-methoxyethoxy)ethoxy]ethoxy]ethoxy]ethoxy]ethoxy]ethoxy]ethoxy]ethanol (4.0 g, 9.33 mmol) in CH₂Cl₂ (40 mL) was added TEA (2.83 g, 28.0 mmol, 3.90 mL), DMAP (114 mg, 933 umol) and 4-methylbenzenesulfonyl chloride (2.67 g, 14.0 mmol). The mixture was stirred at 25° C. for 2 hrs. TLC (CH₂Cl₂/MeOH=10/1, R_{f}=0.4) showed the reaction was complete. The reaction mixture was diluted with CH₂Cl₂ (50 mL) and washed with brine (25 mL×2). The combined organic layers were dried over Na₂SO₄, filtered and concentrated under reduced pressure to give a residue as yellow solid, which was purified by column chromatography (SiO₂, CH₂Cl₂/MeOH=1/0 to 10/1) to give 2-[2-[2-[2-[2-[2-[2-[2-(2-methoxyethoxy)ethoxy]ethoxy]ethoxy]ethoxy]ethoxy]ethoxy]ethoxy]ethyl 4-methylbenzenesulfonate (4.2 g, 54.2% yield) as a yellow solid.

¹HNMR(400 MHz, CD₃ OD) δ 7.76 (d, J=8.0 Hz, 2H), 7.41 (br d, J=8.0 Hz, 2H), 4.11 (t, J=4.4 Hz, 2H), 3.54-3.67 (m, 32H), 3.45-3.53 (m, 7H), 2.42 (s, 3H)

LCMS m/z=583.7 (M+H)⁺, Rt=1.218 min

### 3-2. Preparation of (3S,8aS)-3-[[3-[2-[2-[2-[2-[2-[2-[2-[2-(2-methoxyethoxy) ethoxy]ethoxy]ethoxy]ethoxy]ethoxy]ethoxy]ethoxy]ethyl]imidazol-4-yl]methyl]-2,3,6,7,8,8a-hexahydropyrrolo[1,2-a]pyrazine-1,4-dione

To a solution of 2-[2-[2-[2-[2-[2-[2-[2-(2-methoxyethoxy)ethoxy]ethoxy] ethoxy]ethoxy]ethoxy]ethoxy]ethoxy]ethyl 4-methylbenzenesulfonate (4.20 g, 7.21 mmol) and (3S,8aS)-3-(1H-imidazol-5-ylmethyl)-2,3,6,7,8,8a-hexahydropyrrolo[1,2-a]pyrazine-1,4-dione (2.03 g, 8.65 mmol) in DMF (40 mL) was added Cs₂CO₃ (7.05 g, 21.6 mmol). The mixture was stirred at 50° C. for 2 hrs. TLC (CH₂Cl₂/MeOH=20/1, R_{f}=0.2) showed the reaction was complete. The reaction mixture was diluted with CH₂Cl₂ (80 mL) and washed with brine 80 mL (40 mL×2). The combined organic layers were dried over Na₂SO₄, filtered and concentrated under reduced pressure to give a residue. The reaction mixture was concentrated under reduced pressure to give a residue which was purified by prep-HPLC (The reaction mixture was concentrated under reduced pressure to give a residue which was purified by prep-HPLC (column: WELCH XTIMATE™ C18 150*30 mm*5 um; mobile phase: [water(NH₄HCO₃)-ACN]; B%: 0%-90%, 14 min) to give (3S,8aS)-3-[[3-[2-[2-[2-[2-[2-[2-[2-[2-(2-ethoxyethoxy)ethoxy]ethoxy]ethoxy]ethoxy]ethoxy]ethoxy]ethoxy]ethyl] imidazol-4-yl]methyl]-2,3,6,7,8,8a-hexahydropyrrolo[1,2-a]pyrazine-1,4-dione (4.20 g, 89.2% yield, 98.67% purity) as a white solid.

¹HNMR: 400 MHz, CD₃ OD δ 7.63 (s, 1H), 7.04 (s, 1H), 4.31-4.40 (m, 1H), 4.25 (t, J=8.4 Hz, 1H), 4.15 (t, J=4.8 Hz, 2H), 3.76 (t, J=5.2 Hz, 2H), 3.60-3.64 (m, 32H), 3.52-3.55 (m, 2H), 3.35 (s, 3H), 3.27 (dd, J=14.8, 4.0 Hz, 1H), 2.90 (dd, J=14.8, 8.0 Hz, 1H), 2.23-2.34 (m, 1H), 1.88-2.03 (m, 3H).

### Example 4

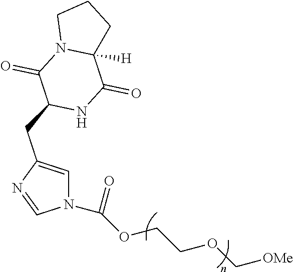

By following the procedure of Example 2, except for employing PEG508 (average molecular weight 508), PEG2000 (n=40.0-50.0, average molecular weight about 2000 Da), PEG3400 (average molecular weight 3400 Da), PEG6000 (average molecular weight 6000 Da), and PEG10000 (average molecular weight 10000 Da) are used in place of 2-[2-[2-[2-[2-[2-[2-[2-(2-methoxyethoxy)ethoxy]ethoxy]ethoxy]ethoxy]ethoxy]ethoxy]ethoxy]ethanol, corresponding PEG modified 4-[[(3S,8aS)-1,4-dioxo-2,3,6,7,8,8a-hexahydropyrrolo[1,2-a]pyrazin-3-yl]methyl]imidazole-1-carboxylate are obtained.

### Example 5

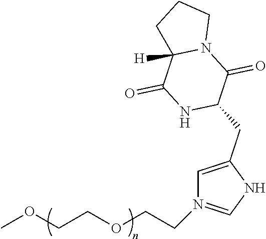

By following the procedure of Example 3-1, Tosyl-PEG1000, Tosyl-PEG2000, Tosyl-PEG3400, Tosyl-PEG6000, Tosyl-PEG10000, Tosyl-PEG2000 are prepared. Then, by following the procedure of Example 3-2, except for employing the Tosyl-PEG1000, Tosyl-PEG2000, Tosyl-PEG3400, Tosyl-PEG6000, Tosyl-PEG10000, Tosyl-PEG2000, in place of 2-[2-[2-[2-[2-[2-[2-[2-(2-methoxyethoxy)ethoxy]ethoxy] ethoxy]ethoxy]ethoxy]ethoxy]ethoxy]ethyl 4-methylbenzenesulfonate, corresponding (3S,8aS)-3-PEG-imidazol-4-yl]methyl]-2,3,6,7,8,8a-hexahydropyrrolo[1,2-a]pyrazine-1,4-diones are obtained.

This invention is described herein with reference to certain embodiments thereof. The scope of this invention is not limited to the embodiments described, but is limited only by the scope of the claims and/or equivalents thereof. Workers of ordinary skill in the art can readily appreciate that other embodiments can be practiced without departing from the scope of this invention. All such variations are considered to be part of this invention.

1. A compound of Formula A′: 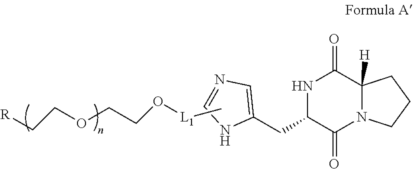
wherein
L₁ is —C(O)—, —S(O)—, —S(O)₂—, —P(O)(O⁻)—, —CH₂C(O)—, —C(O)CH₂—, —C(O)CH₂CH₂—, —CH₂—, —CH₂CH₂—, or —CH₂CH₂CH₂—, said L₁ binds to one of two nitrogen atoms of imidazole group;
n is an integer of 1-500; and
R is hydrogen, a linear or branched C1-C4 alkyl, or a linear or branched C1-C4 alkoxy, or an isomer or a pharmaceutically acceptable salt thereof.

2. The compound of claim 1, wherein the compound of the Formula A′ is a compound of Formula A-1: 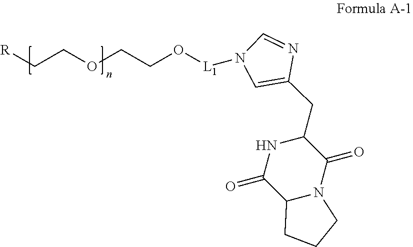
wherein, L₁, n, and R have the same meanings as defined in claim 1.

3. The compound of claim 1, wherein n is 2-50, 3-50, 4-50, 5-50, 6-50, 7-50, 8-50, 9-50, 10-50, 1-40, 2-40, 3-40, 4-40, 5-40, 6-40, 7-40, 8-40, 9-40, 10-40, 1-30, 2-30, 3-30, 4-30, 5-30, 6-30, 7-30, 8-30, 9-30, 10-30, 2-20, 3-20, 4-20, 5-20, 6-20, 7-20, 8-20, 9-20, 10-20, 2-10, 3-10, 4-10, 5-10, 6-10, 7-10, 2-9, 3-9, 4-9, 5-9, 6-9, 7-9, or an isomer or a pharmaceutically acceptable salt thereof.

4. The compound of claim 1, wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or an isomer or a pharmaceutically acceptable salt thereof.

5. The compound of claim 1, wherein L₁ is —C(O)—, —S(O)—, —S(O)₂—, —P(O)(O⁻)—, —CH₂—, or —CH₂CH₂—, or an isomer or a pharmaceutically acceptable salt thereof.

6. The compound of claim 1, wherein R is a linear or branched C1-C4 alkyl, or a linear or branched C1-C4 alkoxy, or an isomer or a pharmaceutically acceptable salt thereof.

7. A composition comprising the compound of claim 1.

8. A modified cyclic dipeptide compound, represented by the following formula (A):
(X₁—X₂)-(L₁)ₚ-O—(CH₂CH₂O)ₙ—(L₂)_{q}—R Formula (A)
wherein
X₁—X₂ is a cyclic dipeptide;
L₁ is a linker selected from C1-C6 alkyl, C1-C6 alkyl-C(O), —C(O)—, —S(O)—, —S(O)₂—, —P(O)(O⁻)—, or C(O)—C1-C6 alkyl;
L₂ is C1-C6 alkyl or C1-C6 alkyl-C(O);
R is hydrogen, a linear or branched C1-C6 alkyl, a linear or branched C1-C6 alkoxy, or a C6-10 aryl;
p is an integer 0 or 1;
q is an integer 0 or 1; and
n is 1 to 500.

9. The modified cyclic dipeptide compound according to claim 8, wherein the cyclic dipeptide is a proline-containing cyclic dipeptide.

10. The modified cyclic dipeptide compound according to claim 8, wherein L₁ is selected from C1-C4 alkyl, C1-C4 alkyl-C(O), —C(O)—, —S(O)—, —S(O)₂—, —P(O)(O⁻)—, or C(O)—C1-C4 alkyl.

11. The modified cyclic dipeptide compound according to claim 8, wherein L₂ is C1-C4 alkyl or C1-C4 alkyl-C(O).

12. The modified cyclic dipeptide compound according to claim 8, wherein the cyclic dipeptide X₁—X₂ is X₁-Pro, X₁-Hyp, Pro-X₂, Hyp-X₂, X₁-His, His-X₂—, wherein X₁ and X₂ are each independently arginine, lysine, histidine, phenylalanine, tyrosine, leucine, isoleucine, methionine, valine, alanine, glycine, proline, glutamic acid, glutamine, serine, threonine, aspartic acid, asparagine, tryptophan, cysteine.

13. The modified cyclic dipeptide compound according to claim 8, wherein the cyclic dipeptide is selected from the group consisting of cyclo(Gly-Pro), cyclo(Phe-Pro), cyclo(Pro-Trp), cyclo(Leu-Pro), cyclo(His-Pro), cyclo(Pro-Asp), cyclo(Pro-Lys), cyclo(Pro-Tyr), cyclo(Pro-Gly), cyclo(Pro-Hyp), cyclo(Pro-Ala), cyclo(Pro-Arg), cyclo(Pro-Leu), cyclo(Hyp-Gly), cyclo(His-Leu), cyclo(Vla-His), cyclo(Met-His), cyclo(Ile-His), cyclo(His-Tyr), cyclo(His-Phe), cyclo(Gln-His), cyclo(Ala-Leu), cyclo(Trp-His), cyclo(Gly-His), cyclo(Thr-His), cyclo(Asp-His), cyclo(Asn-His), cyclo(Arg-His), and a combination thereof, or a salt thereof, or an isomer thereof.

14. The modified cyclic dipeptide compound according to claim 9, wherein L₁ is selected from C1-C4 alkyl, C1-C4 alkyl-C(O), —C(O)—, —S(O)—, —S(O)₂—, —P(O)(O⁻)—, or C(O)—C1-C4 alkyl.
